(19) United States
(12) Patent Application Publication
(10) Pub. No.: US 2009/0137579 A1
(43) Pub. Date: May 28, 2009

(54) Mesylate salt of an IKK inhibitor

(75) Inventors: Frederick A. Hicks, Somerville, MA (US); Martin Ian Cooper, Foxton (GB); Marianne Langston, North Andover, MA (US); Adrian St. Clair Brown, Ely (GB)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 12/288,596

(22) Filed: Oct. 22, 2008

Related U.S. Application Data
(60) Provisional application No. 61/000,012, filed on Oct. 23, 2007.

(51) Int. Cl. A61K 31/5377; C07D 413/14

(52) U.S. Cl. 514/232.5; 544/80

(57) ABSTRACT

The present invention is directed to the compound of formula (II),

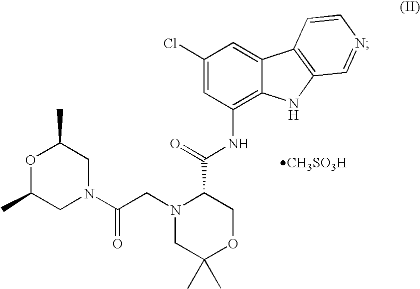

or a solvate thereof, or crystalline forms thereof; to a pharmaceutical composition comprising a pharmaceutically effective amount of the compound of formula (II), including crystalline forms thereof, and a pharmaceutically acceptable carrier; and to the use of a compound of formula (II), or crystalline forms thereof, for treating a patient suffering from, or subject to, a pathological condition capable of being ameliorated by inhibiting IKK-2, and methods related thereto.

### PRIORITY CLAIM

This application claims priority from U.S. Provisional Patent Application No. 61/000,012, filed Oct. 23, 2007, which is hereby incorporated by reference in its entirety.

### FIELD OF THE INVENTION

The present invention is directed to the compound of formula (II):

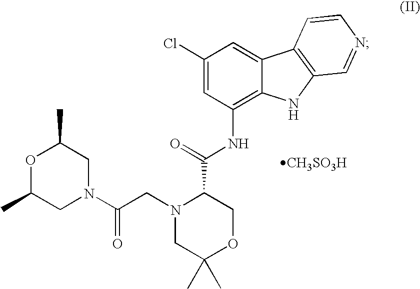

or solvates thereof.

The invention is also directed to the pharmaceutical use of the compound as an IκB inhibitor, crystalline forms thereof, and pharmaceutical compositions comprising the compounds of the invention.

As an inhibitor of IκB kinase, the compound of the invention functions via the selective inhibition of IKK, particularly an IKK-2 inhibitor. Such an inhibitor is particularly useful for treating a patient suffering from or subject to IKK-2 mediated pathological diseases or conditions, e.g., joint inflammation (e.g., rheumatoid arthritis (RA), rheumatoid spondylitis, gouty arthritis, traumatic arthritis, rubella arthritis, psoriatic arthritis, osteoarthritis, and other arthritic conditions), acute synovitis, tuberculosis, atherosclerosis, muscle degeneration, cachexia, Reiter's syndrome, endotoxaemia, sepsis, septic shock, endotoxic shock, gram negative sepsis, gout, toxic shock syndrome, pulmonary inflammatory diseases (e.g., asthma, acute respiratory distress syndrome, chronic obstructive pulmonary disease, silicosis, pulmonary sarcoidosis, and the like), bone resorption diseases, reperfusion injuries, carcinoses, leukemia, sarcomas, lymph node tumors, skin carcinoses, apoptosis, graft versus host reaction, graft versus host disease (GVHD), allograft rejection, leprosy, viral infections (e.g., HIV, cytomegalovirus (CMV), influenza, adenovirus, the Herpes group of viruses, and the like), parasitic infections (e.g., malaria, such as cerebral malaria), yeast and fungal infections (e.g., fungal meningitis), fever and myalgias due to infection, acquired immune deficiency syndrome (AIDS), AIDS related complex (ARC), cachexia secondary to infection or malignancy, cachexia secondary to AIDS or cancer, keloid and scar tissue formation, pyresis, diabetes, inflammatory bowel diseases (IBD) (e.g., Crohn's disease and ulcerative colitis), multiple sclerosis (MS), ischemic brain injury, e.g. cerebral infarction (stroke), head trauma, psoriasis, Alzheimer's disease, carcinomatous disorders (potentiation of cytotoxic therapies), cardiac infarct, chronic obstructive pulmonary disease (COPD), COPD exacerbations, acute respiratory distress syndrome (ARDS), and cancer (e.g., lymphoma, such as diffuse large B-cell, primary mediastinal B-cell, and mantle cell; multiple myeloma; osteolytic bone metastasis; head and neck squamous cell cancer; prostate cancer; pancreatic cancer and non-small cell lung cancer), to name a few, that could be ameliorated by the targeted administration of the inhibitor.

### Reported Developments

NF-κB is a heterodimeric transcription factor that regulates the expression of multiple inflammatory genes. NF-κB has been implicated in many pathophysiologic processes including angiogenesis (Koch et al., Nature 1995, 376, 517-519), atherosclerosis (Brand et al., J Clin Inv. 1996, 97, 1715-1722), endotoxic shock and sepsis (Bohrer et al., J. Clin. Inv. 1997, 100 972-985), inflammatory bowel disease (Panes et al., Am J. Physiol. 1995, 269, H1955-H1964), ischemia/reperfusion injury (Zwacka et al., Nature Medicine 1998, 4, 698-704), and allergic lung inflammation (Gosset et al., Int Arch Allergy Immunol. 1995, 106, 69-77). Thus the inhibition of NF-κB by targeting regulatory proteins in the NF-κB activation pathway represents an attractive strategy for generating anti-inflammatory therapeutics due to NF-κB's central role in inflammatory conditions.

The IκB kinases (IKKs) are key regulatory signaling molecules that coordinate the activation of NF-κB. Many immune and inflammatory mediators including TNFα, lipopolysaccharide (LPS), IL-1β, CD3/CD28 (antigen presentation), CD40L, FasL, viral infection, and oxidative stress have been shown to lead to NF-κB activation. Although the receptor complexes that transduce these diverse stimuli appear very different in their protein components, it is understood that each of these stimulation events leads to activation of the IKKs and NF-κB.

The IKK complex appears to be the central integrator of diverse inflammatory signals leading to the phosphorylation of IκB. Cell and animal experiments indicate that IKK-2 is a central regulator of the pro-inflammatory role of NF-κB, wherein the IKK-2 is activated in response to immune and inflammatory stimuli and signaling pathways. Many of those immune and inflammatory mediators, including IL-1β, LPS, TNFα, CD3/CD28 (antigen presentation), CD40L, FasL, viral infection, and oxidative stress, play an important role in respiratory diseases. Furthermore, the ubiquitous expression of NF-κB, along with its response to multiple stimuli means that almost all cell types present in the lung are potential targets for anti-NF-κB/IKK-2 therapy. This includes alveolar epithelium, mast cells, fibroblasts, vascular endothelium, and infiltrating leukocytes, including neutrophils, macrophages, lympophocytes, eosinophils and basophils.

Inhibitors of IKK-2 are believed to display broad anti-inflammatory activity by inhibiting the expression of genes such as cyclooxygenase-2 and 12-lipoxygenase (synthesis of inflammatory mediators), TAP-1 peptide transporter (antigen processing), MHC class I H-2K and class II invariant chains (antigen presentation), E-selectin and vascular cell adhesion molecule (leukocyte recruitment), interleukins-1,2,6,8 (cytokines), RANTES, eotaxin, GM-CSF (chemokines), and superoxide dismutase and NADPH quinone oxidoreductase (reactive oxygen species).

NF-κB is activated beyond its normal extent in diseases such as rheumatoid arthritis, osteoarthritis, asthma, chronic obstructive pulmonary disease (COPD), rhinitis, multiple sclerosis, cardiac infarction, Alzheimer's diseases, diabetes Type II, psoriasis, inflammatory bowel disease or atherosclerosis.

The inhibition of NF-κB is also described as being useful for treating hypoproliferative diseases, e.g., solid tumor and leukemias, on its own or in addition to cytostatic therapy. Inhibition of the NF-κB-activating signal chain at various points or by interfering directly with the transcription of the gene by glucocorticoids, salicylates or gold salts, has been shown as being useful for treating rheumatism.

Patent applications WO04/092167, US2004-0235839, WO05/111037 and US2005-0239781 disclose beta carboline compounds that exhibit an inhibitory effect on IKK. These applications additionally disclose methods for the preparation of these compounds, pharmaceutical compositions containing these compounds, and methods for the prophylaxis and therapy of diseases, disorders, or conditions associated with an increased activity of IκB kinase, including but not limited to rheumatoid arthritis and multiple sclerosis.

(S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (I) is also specifically disclosed:

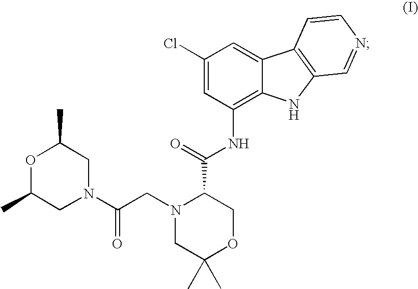

The structure and synthesis of the free-base amorphous form of this compound is provided in the working examples in WO04/092167, US2004-0235839, WO05/111037 and US2005-0239781, and only a general discussion of a wide variety of salts is disclosed. These applications do not disclose specific salts or crystalline forms of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide.

The large-scale manufacturing of a pharmaceutical composition poses many challenges to the chemist and chemical engineer. While many of these challenges relate to the handling of large quantities of reagents and control of large-scale reactions, the handling of the final product poses special challenges linked to the nature of the final active product itself. Not only must the product be prepared in high yield, be stable, and capable of ready isolation, the product must possess properties that are suitable for the types of pharmaceutical preparations in which they are likely to be ultimately used. The stability of the active ingredient of the pharmaceutical preparation must be considered during each step of the manufacturing process, including the synthesis, isolation, bulk storage, pharmaceutical formulation and long-term formulation. Each of these steps may be impacted by various environmental conditions of temperature and humidity.

The pharmaceutically active substance used to prepare the pharmaceutical compositions should be as pure as possible and its stability on long-term storage must be guaranteed under various environmental conditions. These properties are absolutely essential to prevent the appearance of unintended degradation products in pharmaceutical compositions, which degradation products may be potentially toxic or result simply in reducing the potency of the composition.

A primary concern for the manufacture of large-scale pharmaceutical compounds is that the active substance should have a stable crystalline morphology to ensure consistent processing parameters and pharmaceutical quality. If an unstable crystalline form is used, crystal morphology may change during manufacture and/or storage resulting in quality control problems, and formulation irregularities. Such a change may affect the reproducibility of the manufacturing process, and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” While polymorphs of a given substance have the same chemical composition, they may differ from each other with respect to one or more physical properties, such as solubility and dissociation, true density, melting point, crystal shape, compaction behavior, flow properties, and/or solid state stability.

As described generally above, the polymorphic behavior of drugs can be of great importance in pharmacy and pharmacology. The differences in physical properties exhibited by polymorphs affect practical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when it is one polymorph than when it is another polymorph) or mechanical changes (e.g., tablets crumble on storage as a kinetically favored polymorph converts to thermodynamically more stable polymorph) or both (e.g., tablets of one polymorph are more susceptible to breakdown at high humidity). In addition, the physical properties of the crystal may be important in processing: for example, one polymorph might be more likely to form solvates that cause the solid form to aggregate and increase the difficulty of solid handling, or might be difficult to filter and wash free of impurities (i.e., particle shape and size distribution might be different between one polymorph relative to other).

While drug formulations having improved chemical and physical properties are desired, there is no predictable means for preparing new drug forms (e.g., polymorphs) of existing molecules for such formulations. These new forms would provide consistency in physical properties over a range of environments common to manufacturing and composition usage. More particularly, there is a need for an inhibitor of IκB kinase that operates through the selective inhibition of IKK, particularly an IKK-2 inhibitor. Such an inhibitor should have utility in treating a patient suffering from or subject to IKK-2 mediated pathological (diseases) conditions, e.g., rheumatoid arthritis or multiple sclerosis, as well as having properties suitable for large-scale manufacturing and formulation.

In the instant case, no art discloses or teaches a mesylate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, or crystalline forms thereof. More particularly, no art discloses or teaches a mesylate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, or crystalline forms thereof, that is particularly useful for large-scale manufacturing, pharmaceutical formulation, and storage.

### SUMMARY OF THE INVENTION

The present invention is directed to the mesylate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, or crystalline forms thereof. Those forms also have properties that are useful for large-scale manufacturing, pharmaceutical formulation, and storage. The present invention also provides pharmaceutical compositions comprising said salt, or crystalline forms thereof; and methods for uses of these salts, or crystalline forms thereof, for the treatment of a variety of diseases, disorders or conditions as described herein.

The present invention shall be more fully discussed with the aid of the following figures and detailed description below.

### BRIEF DESCRIPTION OF THE DRAWINGS

FIG. 1 is a powder X-ray diffractogram for Form 1 of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 2 is the differential scanning calorimetry (DSC) profile for Form 1 of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 3 is the thermal gravimetric analysis (TGA) profile for Form 1 of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 4 is the vapor sorption profile (VSP) for Form 1 of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 5 is a powder X-ray diffractogram for Form 2 (mono-NMP solvate) of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 6 is the differential scanning calorimetry (DSC) profile for Form 2 (mono-NMP solvate) of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

FIG. 7 is the thermal gravimetric analysis (TGA) profile for Form 2 (mono-NMP solvate) of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate.

### DETAILED DESCRIPTION OF THE INVENTION

### Definitions and Abbreviations

As used above, and throughout the description of the invention, the following terms, unless otherwise indicated, shall be understood to have the following meanings.

“Mesylate Salt” is meant to describe the mesylate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, and has the structure of formula (II).

As used herein, “crystalline” refers to a solid having a highly regular chemical structure. In particular, a crystalline Mesylate Salt may be produced as one or more single crystalline forms of the Mesylate Salt. For the purposes of this application, the terms “single crystalline form” and “polymorph” are synonymous; the terms distinguish between crystals that have different properties (e.g., different XRPD patterns, different DSC scan results). Pseudopolymorphs are typically different solvates of a material, and thus their properties differ from one another. Thus, each distinct polymorph and pseudopolymorph of the Mesylate Salt is considered to be a distinct single crystalline form herein.

“Substantially crystalline” refers to Mesylate Salts that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to Mesylate Salts that are at least 70% crystalline. In other embodiments, substantially crystalline refers to Mesylate Salts that are at least 90% crystalline.

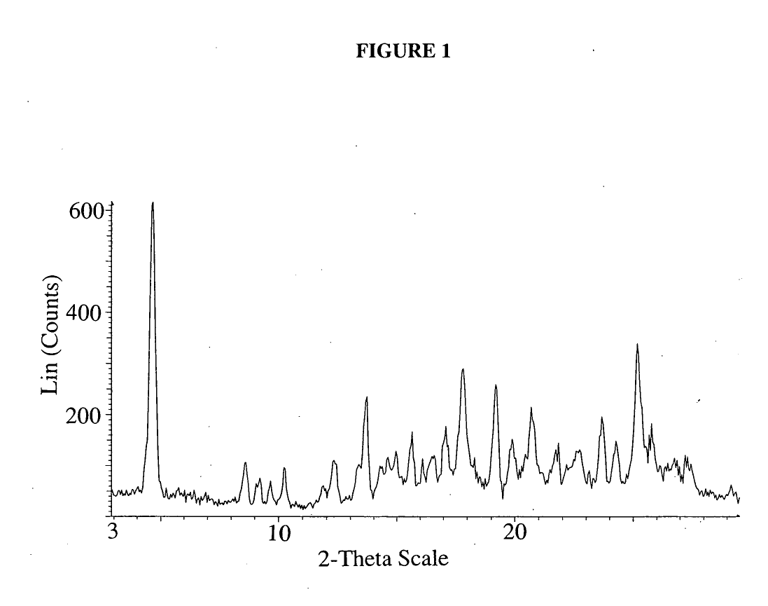

“Form 1” is meant to describe a crystalline form of a compound of formula (II) that may be characterized using distinguishing data. Exemplary data are found in FIGS. 1, 2, 3, and 4, and in Table 1.

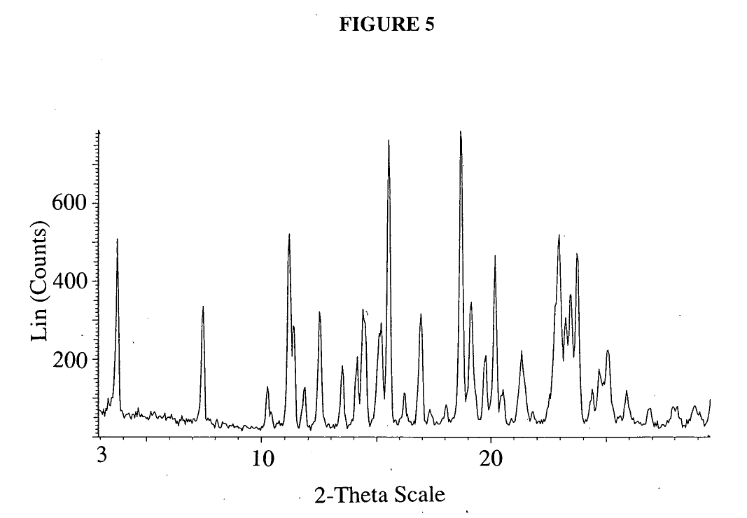

“Form 2” is meant to describe a crystalline form of a compound of formula (II) that may be characterized using distinguishing data. Exemplary data are found in FIGS. 5, 6, and 7, and in Tables 2 and 3.

The term “solvate or solvated” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate.

The term “hydrate” is a solvate wherein the solvent molecule is H₂O that is present in a defined stoichiometric amount, and may for example, include hemihydrate, monohydrate, dihydrate, or trihydrate.

The term “mixture” is used to refer to the combined elements of the mixture regardless of the phase-state of the combination (e.g., liquid or liquid/crystalline).

The term “seeding” is used to refer to the addition of a crystalline material to initiate recrystallization.

The term “antisolvent” is used to refer to a solvent in which compounds of the invention are poorly soluble.

A “subject” is preferably a bird or mammal, such as a human, but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

“Treating” or “treatment” means prevention, partial alleviation, or cure of the disease. The compound and compositions of this invention are useful in treating conditions that are characterized by the activation of NF-κB and/or enhanced levels of cytokines and mediators that are regulated by NF-κB including, but not limited to TNFα and IL-1β. Inhibition or suppression of NF-κB and/or NF-κB-regulated genes such as TNFα may occur locally, for example, within certain tissues of the subject, or more extensively throughout the subject being treated for such a disease. Inhibition or suppression of NF-κB and/or NF-κB-regulated genes such as TNFα may occur by one or more mechanisms, e.g., by inhibiting or suppressing any step of the pathway(s) such as inhibition of IKK.

The term “NF-κB-associated condition” refers to diseases that are characterized by activation of NF-κB in the cytoplasm (e.g., upon phosphorylation of IκB).

The term “TNFα-associated condition” is a condition characterized by enhanced levels of TNFα. In the instant specification, the term NF-κB-associated condition will include a TNFα-associated condition, but is not limited thereto, as NF-κB is involved in the activity and upregulation of other pro-inflammatory proteins and genes.

The term “inflammatory or immune diseases or disorders” is used herein to encompass both NF-κB-associated conditions and TNFα-associated conditions, e.g., any condition, disease, or disorder that is associated with release of NF-κB and/or enhanced levels of TNFα, including conditions as described herein.

“Pharmaceutically effective amount” is meant to describe an amount of a compound, composition, medicament or other active ingredient effective in producing the desired therapeutic effect.

In one aspect, the present invention is directed to the Mesylate Salt of the compound (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide. Accordingly, the present invention provides a compound having structural formula (II):

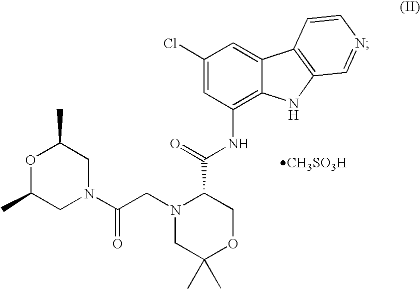

or solvates thereof.

Provided herein is an assortment of characterizing information to describe the Mesylate Salt forms of the compound (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide. It should be understood, however, that not all such information is required for one skilled in the art to determine that such particular form is present in a given composition, but that the determination of a particular form can be achieved using any portion of the characterizing information that one skilled in the art would recognize as sufficient for establishing the presence of a particular form, e.g., even a single distinguishing peak can be sufficient for one skilled in the art to appreciate that such particular form is present.

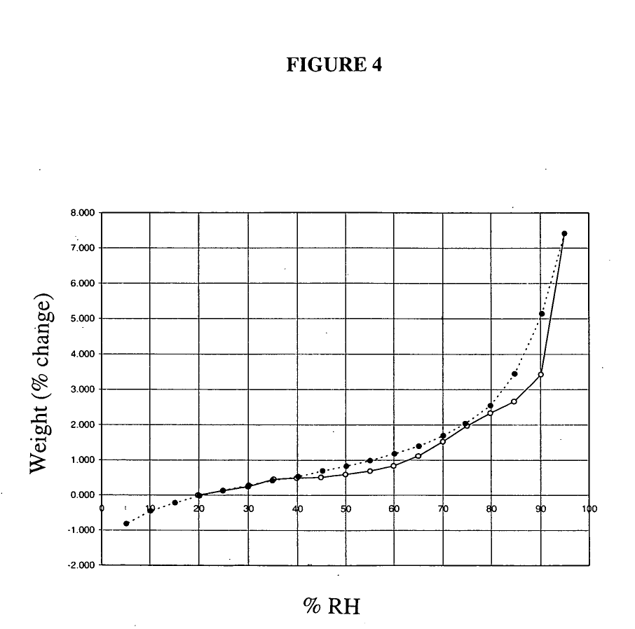

The compound of formula (II) exhibits considerably increased aqueous solubility over the free form. In particular, in water the crystalline free base solubility is about 10 μg/mL and the crystalline Form 1 of the Mesylate Salt has a solubility of greater than about 360 mg/mL. In addition, the compound of formula (II) exhibits low hygroscopicity, in particular, Form 1 of the Mesylate Salt is relatively non-hygroscopic with an uptake of 1.5% at 70% relative humidity (RH) and 3.4% at 90% RH as characterized by the vapor sorption profile for Form 1, shown in FIG. 4.

In some embodiments, the Mesylate Salt is substantially crystalline. Non-limiting examples of crystalline Mesylate Salts include a single crystalline form of the Mesylate Salt (e.g., Form 1); or a mixture of different single crystalline forms (e.g., a mixture of Forms 1 and 2). An embodiment of the invention is also directed to a Mesylate Salt that excludes one or more designated single crystalline forms from a particular weight percentage of the Mesylate (e.g., the Mesylate Salt being at least 90% by weight other than Form 1). Particular weight percentages may be 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%.

Alternatively, embodiments of the invention are directed to a crystalline Mesylate Salt, wherein at least a particular percentage by weight of the crystalline Mesylate Salt is a specific single crystalline form, a combination of particular crystalline forms, or excludes one or more particular crystalline forms. Particular weight percentages may be 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%.

Other embodiments of the invention are directed to the Mesylate Salt being a single crystalline form, or being substantially a designated single crystalline form. The single crystalline form may be a particular percentage by weight of the Mesylate Salt Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. When a particular percentage by weight of a Mesylate Salt is a single crystalline form, the remainder of the Mesylate Salt is some combination of amorphous form of the Mesylate Salt, and one or more crystalline forms of the Mesylate Salt excluding the single crystalline form.

Examples of a single crystalline form include Forms 1 and 2, as well as descriptions of a single crystalline form characterized by one or more properties as discussed herein. The descriptions characterizing the single crystalline forms may also be used to describe the mixture of different forms that may be present in a crystalline Mesylate Salt.

In the following description of particular polymorphs of the Mesylate Salt of the compound (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, embodiments of the invention may be described with reference to a particular crystalline “Form” of the Mesylate Salt (e.g., Form 1 or 2). However, the particular crystalline forms of the Mesylate Salt may also be characterized by one or more of the characteristics of the polymorph as described herein, with or without regard to referencing a particular “Form”.

Form 1

In one embodiment of the invention, a single crystalline form of the Mesylate Salt is characterized as Form 1. Form 1 can be prepared by recrystallization of the Mesylate Salt from ethanol or isopropanol. The recrystallization is effected by dissolution of the Mesylate Salt in the solvent followed by crystallization upon cooling. The dissolution of the Mesylate Salt maybe carried out at ambient temperature or at an elevated temperature, and is preferably carried out at an elevated temperature. One skilled in the art will be able to select a suitable temperature in view of the solvent being used. In some embodiments, the temperature is at least about 40° C., 50° C. or 60° C. In other embodiments, the temperature is less than about 60° C., 70° C., 80° C. or 90° C. Any ranges encompassing these high and low temperatures are included within the scope of the invention. The dissolution is preferably performed at temperatures in the range of about 40° C. to about 90° C., or about 50° C. to about 80° C.

In another embodiment, Form 1 can also be prepared directly by dissolution of the compound of formula (I) in a solvent, followed by contacting the resulting solution with methanesulfonic acid followed by crystallization. The crystallization may be effected with or without seeding. In one embodiment, the solvent is acetone, acetonitrile, 2-butanone (MEK), tetrahydrofuran, 2-methyltetrahydrofuran, or mixtures thereof. In another embodiment, the solvent is aqueous acetone, aqueous acetonitrile, aqueous 2-butanone (MEK), aqueous tetrahydrofuran, aqueous 2-methyltetrahydrofuran, or mixtures thereof. In another embodiment, the solvent is isopropylacetate, nitromethane, toluene, anisole, or mixtures thereof. In another embodiment, the solvent is N,N′-dimethylformamide, N,N′-dimethylacetamide, dimethylsulfoxide, or mixtures thereof, with methyl isobutyl ketone, methyl tert-butyl ether, or mixtures thereof as an antisolvent.

The dissolution of the compound of formula (I), and the contacting of the solution of compound of formula (I) with methanesulfonic acid are preferably carried out at an elevated temperature, which may be the same or different. Each of the dissolution and the contacting steps may involve several different temperatures or temperature ranges. One skilled in the art will be able to select suitable temperatures in view of the conditions being used. In some embodiments, the temperature is at least about 30° C., 40° C., 50° C. or 60° C. In other embodiments, the temperature is less than about 70° C., 80° C., 90° C. or 100° C. Any ranges encompassing these high and low temperatures are included within the scope of the invention. The temperature is preferably in the range of about 30° C. to about 100° C., or about 50° C. to about 80° C.

In another embodiment, Form 1 of the Mesylate Salt can be characterized by the X-ray powder diffraction (herein referred to as “XRPD”) pattern shown in FIG. 1, and data shown in Table 1, obtained using CuKα radiation. In a particular embodiment of the invention, the polymorph can be characterized by one or more of the peaks taken from FIG. 1.

TABLE 1

| | Relative |
|---|---|
| Angle | Intensity |
| 2-θ° | % |
| 4.619 | 100.0 |
| 8.573 | 17.2 |
| 9.187 | 12.1 |
| 9.615 | 11.2 |
| 10.245 | 15.5 |
| 11.863 | 9.7 |
| 12.321 | 17.8 |
| 13.300 | 16.5 |
| 13.652 | 38.0 |
| 14.286 | 16.0 |
| 14.594 | 18.8 |
| 14.946 | 20.7 |
| 15.619 | 26.9 |
| 16.115 | 18.3 |
| 16.573 | 19.4 |
| 17.090 | 28.6 |
| 17.834 | 46.9 |
| 19.225 | 41.9 |
| 19.876 | 24.6 |
| 20.728 | 34.8 |
| 21.803 | 23.3 |
| 22.208 | 16.5 |
| 22.706 | 21.2 |
| 23.711 | 31.7 |
| 24.318 | 23.9 |
| 25.212 | 54.9 |
| 25.792 | 29.4 |
| 26.817 | 18.6 |
| 27.263 | 19.4 |
| 27.500 | 16.7 |
| 29.167 | 10.0 |

In a further particular embodiment, the peaks are identified at 20 angles of 4.619°, 13.652°, 17.834°, 19.225°, 20.728°, 23.711°, and 25.212°. In another further particular embodiment, the peaks are identified at 20 angles of 4.619°, 17.834°, 19.225°, and 25.212°.

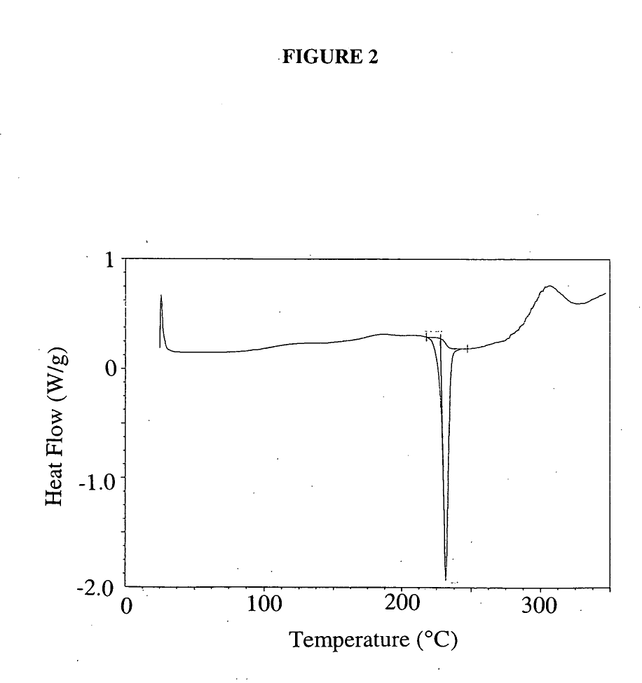

In another embodiment, Form 1 of the Mesylate Salt can be characterized by the differential scanning calorimetry (herein referred to as “DSC”) profile shown in FIG. 2. The profile plots the heat flow as a function of temperature from a sample containing Form 1. The material has a sharp endotherm with an onset temperature of 227.8° C., and a melt at 231.7° C. These temperatures have an error of ±1° C., and are conducted at a temperature scanning rate of 10° C./minute.

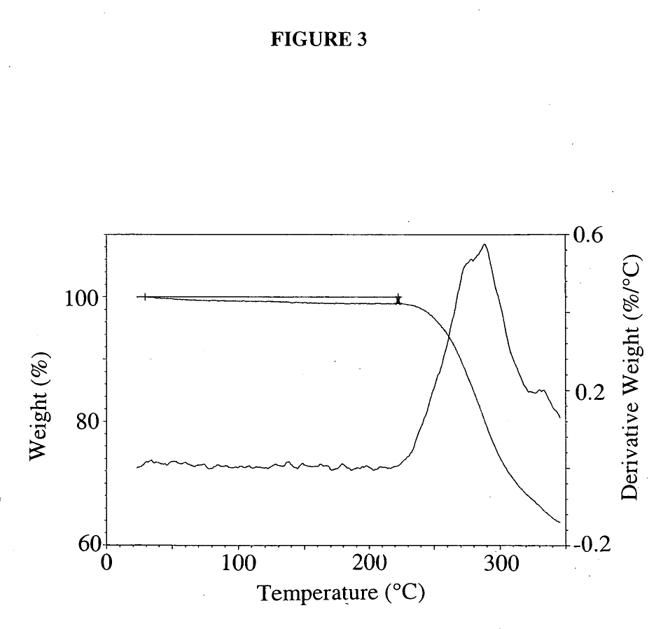

In another embodiment, Form 1 of the Mesylate Salt can be characterized by the thermal gravimetric analysis (herein referred to as “TGA”) profile shown in FIG. 3. The profile graphs the percent loss of weight of the sample as a function of temperature, the temperature rate change being about 10° C./min. There is a weight loss of about 1.044% of the weight of the sample as the temperature is changed from 25° C. to 235° C. This weight loss corresponds with the endotherm of the sample seen in the DSC profile shown in FIG. 2. These temperatures have an error of ±1° C.

In another embodiment, Form 1 of the Mesylate Salt can be characterized by the vapor sorption profile, as shown in FIG. 4. Form 1 is relatively non-hygroscopic with an uptake of 1.5% at 70% relative humidity (RH) and 3.4% at 90% RH. A slight hysteresis was observed along the curve, but the weight gain was reversible.

In another embodiment, Form 1 of the Mesylate Salt is characterized by at least one of the following features (I-i)-(I-iii):
(I-i) at least one of the X-ray powder diffraction peaks shown in Table 1.
(I-ii) an X-ray powder diffraction pattern substantially similar to FIG. 1.
(I-iii) a differential scanning calorimetry (DSC) profile having an endotherm range of about 215° C. to about 250° C.
   In a further embodiment of the invention, Form 1 of the Mesylate Salt is characterized by all of the features (I-i)-(I-iii) above.

Form 2

In another embodiment of the invention, a single crystalline form of the Mesylate Salt is characterized as Form 2. Form 2 is a mono-N-methylpyrrolidinone (NMP) solvate form of the Mesylate Salt. Form 2 is a pseudopolymorph that can be produced by dissolution of the Mesylate Salt in N-methylpyrrolidinone (NMP), and subsequent crystallization.

In another embodiment, Form 2 of the Mesylate Salt can be characterized by the XRPD pattern shown in FIG. 5, and data shown in Table 2, obtained using CuKα radiation. In a particular embodiment of the invention, Form 2 is characterized by one or more of the peaks taken from FIG. 5.

TABLE 2

| | Relative |
|---|---|
| Angle | Intensity |
| 2-θ° | % |
| 3.694 | 64.6 |
| 7.401 | 42.6 |
| 10.258 | 16.3 |
| 11.163 | 66.3 |
| 11.4 | 36 |
| 11.835 | 16.1 |
| 12.529 | 40.8 |
| 13.497 | 23.2 |
| 14.149 | 26.1 |
| 14.45 | 41.7 |
| 15.185 | 37.1 |
| 15.551 | 97.1 |
| 16.237 | 14.3 |
| 16.935 | 40.2 |
| 17.35 | 9 |
| 18.036 | 10.5 |
| 18.737 | 100 |
| 19.123 | 44 |
| 19.767 | 26.6 |
| 20.183 | 59.2 |
| 20.489 | 15.5 |
| 21.356 | 28.1 |
| 23.001 | 66 |
| 23.267 | 39.1 |
| 23.5 | 46.5 |
| 23.776 | 59.9 |
| 24.38 | 15.6 |
| 24.711 | 22.2 |
| 25.091 | 28.4 |
| 25.908 | 15.2 |
| 26.895 | 9.3 |
| 28.093 | 9.9 |
| 28.876 | 10.1 |

In a further particular embodiment, the peaks are identified at 20 angles of 3.694°, 11.163°, 15.551°, 18.737°, 20.183°, 23.001°, and 23.776°.

In another embodiment, a variable temperature analysis showed no change in the pattern of the XRPD at temperatures less than about 150° C.

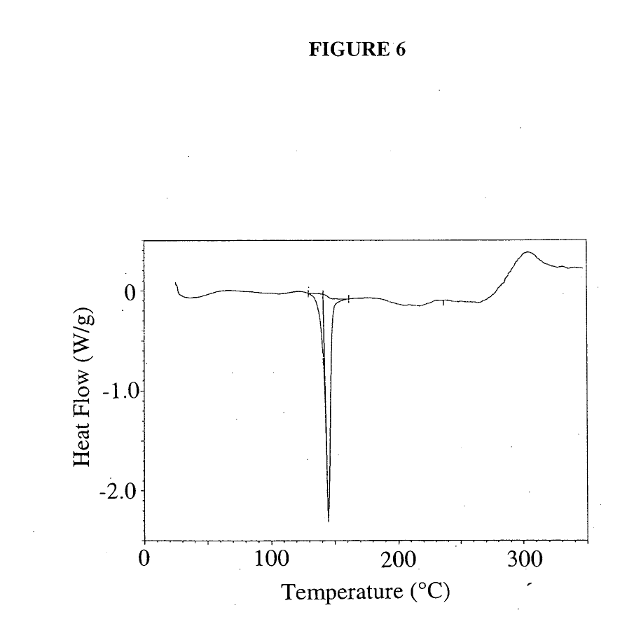

In another embodiment, Form 2 of the Mesylate Salt can be characterized by the differential scanning calorimetry (DSC) profile shown in FIG. 6. The profile plots the heat flow as a function of temperature from a sample containing Form 2. The material has a sharp endotherm with an onset temperature of 140.8° C. and a melt at 144.8° C. These temperatures have an error of ±1° C., and are conducted at a temperature scanning rate of 10° C./minute.

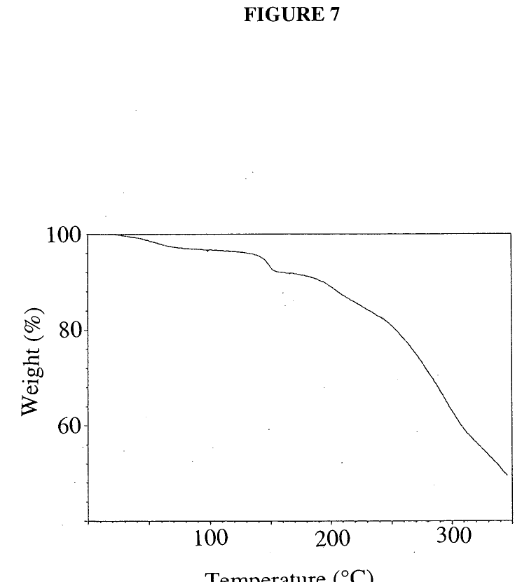

In another embodiment, Form 2 of the Mesylate Salt can be characterized by the thermal gravimetric analysis (TGA) profile shown in FIG. 7. The profile graphs the percent loss of weight of the sample as a function of temperature, the temperature rate change being about 10° C./min. There is a weight loss in 3 stages of about 1.044%, 5.484% and 7.067% as the temperature is changed from 25° C. to 235° C. This first weight loss corresponds with a small nonsolvated solvent loss, probably water, and the 2^{nd} and 3^{rd} losses correspond to the melting endotherm of the sample and slow loss of the solvate NMP. These temperatures have an error of ±1° C.

In another embodiment, Form 2 of the Mesylate Salt can be characterized by at least one of the following features (II-i)-(II-iii):
(II-i) at least one of the X-ray powder diffraction peaks shown in Table 2.
(II-ii) an X-ray powder diffraction pattern substantially similar to FIG. 5.
(II-iii) a differential scanning calorimetry (DSC) profile showing a endotherm range of about 120° C. to about 170° C.

In a further embodiment of the invention, Form 2 of the Mesylate Salt can be characterized by all of the features (II-i)-(II-iii) above.

In another embodiment, Form 2 of the Mesylate Salt can be characterized by the single crystal X-Ray diffraction (SCXRD) data shown in Table 3 below. A good correlation was obtained between the experimental and calculated values.

The structure solution was obtained by direct methods, full-matrix least-squares refinement on F² with weighting w⁻¹=σ² (Fₒ²)+(0.0760 P)²+(5.0000 P), where P=(Fₒ²+2F_{c}²)/3, anisotropic displacement parameters, no absorption correction, absolute structure parameter=0.03(5). Final wR²={Σ[w(Fₒ²−F_{c}²)²]/Σ[w(Fₒ²)²]^{1/2}}=0.181 for all data, conventional R₁=0.0527 on F values of 6049 reflections with Fₒ>4σ(Fₒ), S=1.007 for all data and 439 parameters. Final Δ/σ(max) 0.001, Δ/σ(mean), 0.000. A final difference map between +0.857 and −0.624e.Å⁻³.

TABLE 3

| Molecular formula | C₃₂H₄₅ClN₆O₈S | | | | |
|---|---|---|---|---|---|
| Molecular weight | 709.25 | | | | |
| Crystal system | n/a | | | | |
| Space group | P21 | a | 8.6229(5) Å | α | 90° |
| | | b | 8.7356(2) Å | β | 98.6930(11)° |
| | | c | 23.7689(7) Å | γ | 90° |

| | |
|---|---|
| V | 1769.85(12) Å³ |
| Z | 2 |
| D_{c} | 1.331 g · cm⁻¹ |
| μ | 0.224 mm⁻¹ |
| Source, λ | Mo—K(alpha), 0.71073 Å |
| F(000) | 752 |
| T | 120(2) K |
| Crystal | 0.32 × 0.28 × 0.12 mm |
| Data truncated to | 0.80 Å |
| θₘₐₓ | 26.37° |
| Completeness | 97.6% |
| Reflections | 8090 |
| Unique reflections | 6259 |
| Rᵢₙₜ | 0.0449 |

Pharmaceutical Compositions and Methods

The pharmacological properties of the compound of formula (II), or crystalline forms thereof, are such that it is suitable for use in the treatment of all those patients suffering from or subject to conditions that can be ameliorated by the administration of an inhibitor of IκB kinase.

In yet another aspect, a method for treating an inflammatory disease or immune-related disease is provided comprising administering a pharmaceutically effective amount of the compound of formula (II), including crystalline forms thereof, or a pharmaceutical composition thereof, to a subject in need thereof. In still another aspect, a method for treating cancer is provided comprising administering a pharmaceutically effective amount of the compound of formula (II), including crystalline forms thereof, or a pharmaceutical composition thereof, to a subject in need thereof.

More particularly, the present compounds are useful for treating or lessening the severity of an inflammatory disease, an immune-related disease or cancer. In some embodiments, these diseases and disorders include, but are not limited to, joint inflammation (e.g., rheumatoid arthritis (RA), rheumatoid spondylitis, gouty arthritis, traumatic arthritis, rubella arthritis, psoriatic arthritis, osteoarthritis, and other arthritic conditions), acute synovitis, tuberculosis, atherosclerosis, muscle degeneration, cachexia, Reiter's syndrome, endotoxaemia, sepsis, septic shock, endotoxic shock, gram negative sepsis, gout, toxic shock syndrome, pulmonary inflammatory diseases (e.g., asthma, acute respiratory distress syndrome, chronic obstructive pulmonary disease, silicosis, pulmonary sarcoidosis, and the like), bone resorption diseases, reperfusion injuries, carcinoses, leukemia, sarcomas, lymph node tumors, skin carcinoses, lymphoma, apoptosis, graft versus host reaction, graft versus host disease (GVHD), allograft rejection, leprosy, viral infections (e.g., HIV, cytomegalovirus (CMV), influenza, adenovirus, the Herpes group of viruses, and the like), parasitic infections (e.g., malaria, such as cerebral malaria), yeast and fungal infections (e.g., fungal meningitis), fever and myalgias due to infection, acquired immune deficiency syndrome (AIDS), AIDS related complex (ARC), cachexia secondary to infection or malignancy, cachexia secondary to AIDS or cancer, keloid and scar tissue formation, pyresis, diabetes, inflammatory bowel diseases (IBD) (e.g., Crohn's disease and ulcerative colitis), multiple sclerosis (MS), ischemic brain injury, e.g. cerebral infarction (stroke), head trauma, psoriasis, Alzheimer's disease, carcinomatous disorders (potentiation of cytotoxic therapies), cardiac infarct, chronic obstructive pulmonary disease (COPD), COPD exacerbations, and acute respiratory distress syndrome (ARDS). In other embodiments, compounds of the invention are useful for treating cancer, especially for treating cancers where IKK activity is abnormally high. The cancer types that may be treated include lymphoma, such as diffuse large B-cell (Davis, et al., J. Exp. Med. 2001, 194, 1861-1874; Lam et al., Clin. Cancer Res. 2005, 11, 2840; Feuerhake et al., Blood 2005, 106, 1392-1399), primary mediastinal B-cell, and mantle cell; multiple myeloma (Berenson et al., Clin. Adv. Hematol. Oncol. 2004, 2, 162-166; Gunn et al., Stem Cells, 2005); osteolytic bone metastasis (Ruocco et al., J. Exp. Med. 2005, 201, 1677-1687; Morony et al., Endocrinology 2005, 146, 3235-3243; Gordon, et al., Cancer Res. 2005, 65, 3209-3217; RoleSohara et al., Cancer Lett. 2005, 228, 203-209); head and neck squamous cell cancer (van Hogerlinden et al., J. Invest. Dermatol. 2004, 123 101-108; Tamatani et al, Int. J. Cancer 2004, 108, 912-921; Loercher et al., Cancer Res. 2004, 64, 6511-6523; Van Waes et al., Int. J. Radiat. Oncol. Biol. Phys. 2005, 63, 1400-1412); prostate cancer; pancreatic cancer and non-small cell lung cancer. In some embodiments, the compound of formula (II), or crystalline forms thereof, is useful for treating inflammatory and immune-related diseases, disorders and symptoms, more especially, inflammatory ones such as RA, asthma, IBD, psoriasis, psoriatic arthritis, COPD, COPD exacerbations and MS. In some embodiments, the compound of formula (II), or crystalline forms thereof, is useful for treating inflammatory and immune-related diseases, disorders and symptoms, more especially, inflammatory ones such as RA, IBD, psoriasis, COPD and COPD exacerbations. In a further embodiment, the compound of formula (II), or crystalline forms thereof, is useful for treating inflammatory and immune-related diseases, disorders and symptoms, more especially, inflammatory ones such as RA.

It will also be appreciated that the compound of formula (II), or crystalline forms thereof, is useful for treating diseases, disorders or symptoms related to the activity of NF-κB, TNF-α, and other enzymes in pathways where IKK is known to modulate activity.

Accordingly, in another aspect of the present invention, pharmaceutical compositions are provided, wherein these compositions comprise the compound of formula (II), or a crystalline form thereof, and a pharmaceutically acceptable carrier. In certain embodiments, these compositions optionally further comprise one or more additional therapeutic agents.

As described above, the pharmaceutically acceptable compositions of the present invention additionally comprise a pharmaceutically acceptable carrier, which, as used herein, includes any and all solvents, diluents, or other liquid vehicle, dispersion or suspension aids, gelatin or polymeric capsule shell, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compound of formula (II), or crystalline forms thereof, or a pharmaceutical composition thereof, according to the method of the present invention, may be administered using any amount and any route of administration effective for treating the disease. The exact amount required will vary from subject to subject, depending on the species, age, and general condition of the subject, the severity of the infection, the particular agent, its mode of administration, and the like. The compound of formula (II), or crystalline forms thereof, or a pharmaceutical composition thereof, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The compound of formula (II), or crystalline forms thereof, or a pharmaceutical composition thereof, can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. Alternatively, compositions for rectal or vaginal administration are gels or creams that can be prepared by mixing compounds with suitable non-irritating excipients such as oils or water to solubilize the compound and polymers and fatty alcohols can be added to thicken the formulation to increase the residual time in the rectal or vaginal cavity and release the active compound.

Solid dosage forms for oral administration include capsules, tablets, pills, powders, and granules. In such solid dosage forms, the active compound may optionally be mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In other embodiments, the active compound may be encapsulated in a gelatin or polymeric capsule shell without any additional agents (neat capsule shell).

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. The solid dosage forms may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

While the compound of formula (II), or crystalline forms thereof, may be used in an application of monotherapy to treat a disorder, disease or symptom, it also may be used in combination therapy, in which the use of an inventive compound or composition (therapeutic agent) is combined with the use of one or more other therapeutic agents for treating the same and/or other types of disorders, symptoms and diseases. Combination therapy includes administration of the therapeutic agents concurrently or sequentially. Alternatively, the therapeutic agents can be combined into one composition which is administered to the patient.

In one embodiment, the compound of formula (II), or crystalline forms thereof, is used in combination with other therapeutic agents, such as other inhibitors of IKK, other agents useful in treating NF-κB and TNF-α associated conditions, and agents useful for treating other disorders, symptoms and diseases. In particular, agents that induce apoptosis such as agents that disrupt cell cycle or mitochondrial function are useful in combination with the IKK inhibitors of this invention. Exemplary agents for combination with the IKK inhibitors include antiproliferative agents (e.g., methotrexate) and the agents disclosed in U.S. Pat. Application Publication No. US2003/0022898, p 14, para. [0173-0174], which is incorporated herein in its entirety. In some embodiments, the compound of the invention is administered in conjunction with a therapeutic agent selected from the group consisting of cytotoxic agents, radiotherapy, and immunotherapy. Non-limiting examples of cytotoxic agents suitable for use in combination with the IKK inhibitors of the invention include capecitibine; gemcitabine; irinotecan; fludarabine; 5-fluorouracil or 5-fluorouracil/leucovorin; taxanes, including, e.g., paclitaxel and docetaxel; platinum agents, including, e.g., cisplatin, carboplatin, and oxaliplatin; anthracyclins, including, e.g., doxorubicin and pegylated liposomal doxorubicin; mitoxantrone; dexamethasone; vincristine; etoposide; prednisone; thalidomide; herceptin; temozolomide; and alkylating agents such as melphalan, chlorambucil, and cyclophosphamide. It is understood that other combinations may be undertaken while remaining within the scope of the invention.

The preparation and properties of the compounds of the invention are described in the following experimental section.

### EXAMPLES

### Example 1

### Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide Mesylate Form 1

(S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (17.273 kg, 33.6 mol, 1.0 equiv.) was suspended in 2-butanone (107 L) and water (2.4 L) in a 160 L reactor followed by heating to 80° C. to provide a solution. Filtration (preheated 0.2 micron inline filter) was followed by washing with 2-butanone (12 L) and the addition of methanesulfonic acid (951 g, 9.9 mol) at 60° C. Seeding with (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide methanesulfonate (168 g) slurried in 2-butanone (1 L) and water (24 mL) gave a brown-yellow solution. Addition of the remaining methanesulfonic acid (2.261 kg, 23.53 mol) via a dose system at 65° C. over 6 h was started. After 1 h 17 min of addition, more seed crystals (52 g) were added, this time giving a suspension. After completion of methanesulfonic add addition and stirring for an additional 3.5 h at 60° C., the suspension was cooled to 0° C. over 3 h 45 min and stirred at 0° C. for 3 h 15 min. Filtration was followed by the washing of the reactor and the filter cake with 2-butanone (12 L). The yellow solid was dried under nitrogen for 3 h, followed by drying in a Provatech dryer overnight at 50° C. to provide 16.076 kg (78%) of the title compound as yellow crystals.

### Example 2

### Preparation of Form 1 of the Mesylate Salt from Other Solvents

Form 1 of the Mesylate Salt can also be produced directly from other solvents following the general method outlined in Example 1, using the specific conditions outlined in Table 4 below. “Acid” in Table 4 refers to methanesulfonic acid.

TABLE 4

| Solvent | Conditions |
|---|---|
| Acetone | Acid addition and crystallization at 50° C. |
| | With or without added water |
| Acetonitrile | Acid addition and crystallization at 50° C. |
| | With or without added water |
| Tetrahydrofuran | Acid addition and crystallization at 50-60° C. |
| 2-Methyltetrahydrofuran | Acid addition and crystallization at 60° C. |
| Toluene | Acid addition and crystallization at 50° C. |
| Anisole | Acid addition and crystallization at 60° C. |
| Isopropyl acetate | Acid addition and crystallization at 50° C. |
| Nitromethane | Acid addition at 50° C., crystallization |
| | at 20° C. |
| N,N-Dimethylformamide | Methyl isobutyl ketone or methyl tert- |
| | butyl ether as antisolvent |
| N,N-Dimethylacetamide | Methyl isobutyl ketone or methyl tert- |
| | butyl ether as antisolvent |
| Dimethylsulfoxide | Methyl isobutyl ketone or methyl tert- |
| | butyl ether as antisolvent |

### Example 3

### Preparation of (S)—N-(6-chloro-9H-pyrido[3,4-b]indol-8-yl) (2-((2S,6R)-2,6-dimethylmorpholino)-2-oxoethyl)-6,6-dimethylmorpholine-3-carboxamide Mesylate Form 1

(S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate can be recrystallized from ethanol or isopropanol to produce Form 1 of the Mesylate Salt using the conditions outlined in Table 5 below.

TABLE 5

| Solvent | Conditions |
|---|---|
| Ethanol | Crystallization after dissolution in ethanol |
| | at 50° C. and cooling |
| Isopropanol | Crystallization after dissolution in |
| | isopropanol at 50° C. and cooling |

### Example 4

### Solubility

The water solubility of Mesylate Salt Form 1 was measured at ambient temperature. Table 6 is a summary of the equilibrium solubility. For Form 1, the solubility is much greater than the free base which has an intrinsic solubility of ˜10 μg/mL.

TABLE 6

| Salt | Solubility (mg/mL) | pH |
|---|---|---|
| Mesylate Form 1 | >360 | 2.52 |

### Example 5

### Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide Mesylate Mono-NMP Solvate, Form 2

A reaction vessel was charged with of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mesylate (400 mg, 0.656 mmol) and N-methylpyrrolidinone (0.8 mL). The solution was stirred at ambient temperature for 14 h followed by seeding and further stirring for 3 h. The material was isolated by filtration to provide (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide methanesulfonate mono-N-methylpyrrolidinone (NMP) solvate, Form 2, after drying.

### Example 6

### X-Ray Powder Diffractometry (XRPD)

X-ray powder diffraction patterns for the samples are acquired on a Bruker AXS D8Advance diffractometer. The data are collected over an angular range of 2.9° to 29.6° 2θ in continuous scan mode using a step size of 0.05° 2θ and a step time of 2 seconds. The sample is run under ambient conditions and prepared as a flat plate specimen using powder as received without grinding. Data for Form 1 are depicted in FIG. 1 and Table 1, and data for Form 2 are depicted in FIG. 5 and Table 2.

### Example 7

### Differential Scanning Calorimetry (DSC)

Differential scanning calorimetry (DSC) data are collected on a TA Instruments Q100 differential scanning calorimeter equipped with a 50 position auto-sampler. The energy and temperature calibration standard is indium. Samples are heated at a rate of 10° C. per minute between 25° C. and 300° C. A nitrogen purge flowing at 50 mL per minute is maintained over the sample during a scan. Between 1 mg and 3 mg of sample is analyzed. All samples are crimped in a hermetically sealed aluminum pan with a pinhole to alleviate the pressure accumulated from the solvent vapor. Data for Form 1 are depicted in FIG. 2 and data for Form 2 are depicted in FIG. 6.

### Example 8

### Thermal Gravimetric Analysis (TGA)

Thermal gravimetric analysis (TGA) data are collected on a TA Instruments Q500 thermal gravimetric analyzer, calibrated with Nickel/Alumel and running at a scan rate of 10° C. per minute. A nitrogen purge flowing at 60 mL per minute is maintained over the sample during measurements. Typically 10 mg to 15 mg of sample is loaded onto a pre-tared platinum crucible. Data for Form 1 are depicted in FIG. 3 and data for Form 2 are depicted in FIG. 7.

### Example 9

### Gravimetric Vapor Sorption (GVS)

Gravimetric vapor sorption (GVS) data are collected using a SGA-100 Water Vapor Sorption Analyzer from VTI Corporation. Sample sizes are typically 10 mg. A moisture adsorption/desorption isotherm is recorded by subjecting samples to a series of relative humidity (RH) steps. Data for Form 1 are depicted in FIG. 4.

### Example 10

### Single Crystal X-Ray Diffraction (SCXRD)

Single crystal X-Ray

Diffraction data are collected using a Bruker AXS 1K SMART CCD diffractometer equipped with an Oxford Cryosystems Cryostream cooling device. The structures were solved using either the SHELXS or SHELXD programs and refined with the SHELXL program as part of the Bruker AXS SHELXTL suite. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter. Data for Form 2 are found in Table 3.

### Example 11

### Biological Testing

Compounds of this invention are effective inhibitors of IκB kinase (IKK), and therefore, are useful for treating conditions caused or aggravated by the activity of this kinase. The in vitro and in vivo IκB kinase inhibitory activities of the compounds of formula (I) and (II) may be determined by various procedures known in the art. The potent affinities for IκB kinase exhibited by the inventive compounds can be measured as an IC₅₀ value (in nM), which is the concentration (in nM) of compound required to provide 50% inhibition of IκB kinase.

Assay for Measuring IκB Kinase Enzyme Inhibition

An in vitro assay for detecting and measuring inhibition activity against IκB kinase complex by candidate pharmacological agents can employ a biotinylated GST fusion protein spanning residues 5-55 of IκBA (SwissProt Accession No. P25963, Swiss Institute of Bioinformatics, Geneva, Switzerland) and an agent for detection of the phosphorylated product, e.g. a specific antibody binding only to the phosphorylated form GS, being either monoclonal or polyclonal (e.g., commercially-available anti-phospho-serine³² IκB antibodies). In the example of detecting the phosphorylated product by an anti-phosphoserines^{32 and 36} IκB antibody, once the antibody-phospho-GST-IκBα complex is formed, the complex can be detected by a variety of analytical methods (e.g., radioactivity, luminescence, fluorescence, or optical absorbance). For the use of the time resolved fluorescence method the antibody is labeled with europium chelate and the antibody-phospho-GST-IκBα complex is bound to biotin binding protein conjugated to a fluorescence acceptor (e.g., Steptavidin Alexa647, Invitrogen, Carlsbad, Calif.). How to prepare materials for and conduct this assay are described in more detail below.

Isolation of the IκB Kinase Complex

An IκB-α kinase complex is prepared by first diluting 10 ml of HeLa S3 cell-extracts S100 fraction (Lee et al., Cell 1997, 88, 213-222) with 40 ml of 50 mM HEPES pH 7.5. Then, 40% ammonium sulfate is added and incubated on ice for 30 minutes. The resulting precipitated pellet is redissolved with 5 ml of SEC buffer (50 mM HEPES pH 7.5, 1 mM DTT, 0.5 mM EDTA, 10 mM 2-glycerophosphate), clarified by centrifugation at 20,000×g for 15 min., and filtrated through a 0.22 μm filter unit. The sample is loaded onto a 320 ml SUPEROSE-6 gel filtration FPLC column (Amersham Biosciences AB, Uppsala, Sweden) equilibrated with a SEC buffer operated at 2 ml/min flow rate at 4° C. Fractions spanning the 670-kDa molecular-weight marker are pooled for activation. A kinase-containing pool is then activated by incubation with 100 nM MEKK1Δ (Lee et al., Cell 1997, 88, 213-222) 250 μM MgATP, 10 mM MgC₂, 5 mM DTT, 10 mM 2-glycerophosphate, 2.5 μM Microcystin-LR, for 45 minutes at 37° C. The activated enzyme is stored at −80° C. until further use.

Measurement of IκB Kinase Phospho-Transferase Activity

To each well of a 384 well plate, compounds of various concentrations in 1 μL of DMSO are incubated for 2 hours with 30 μL of assay buffer (50 mM Hepes pH 7.5, 5 mM DTT, 10 mM MgCl₂ 10 mM 2-glycerophosphate, 0.1% Bovine Serum Albumin) containing a 1:90 dilution of activated enzyme, 100 nM biotinylated-GST-IκBα 5-55, and 50 μM ATP. Reactions are quenched with the addition of 10 μL of 250 mM EDTA before the addition of 40 μL of detection buffer (50 mM Hepes pH 7.5, 0.1% Bovine Serum Albumin, 0.01% Tween20, Pierce, Rockford, Ill.) containing 2 nM europium labeled anti-IκBα phosphoserine^{32 and 36} and 0.003 mg/mL Streptavidin Alexa647. Samples are allowed to incubate for 1 hour prior to reading on a Wallac Victor plate reader (Perkin Elmer Life and Analytical Sciences, Boston, Mass.). As the assay has been previously shown to be linear with respect to enzyme concentration and time at the enzyme dilution tested, levels of time resolved fluorescence energy transfer are used to determine the inhibition activity of candidate pharmacological agents.

The compounds of the invention are inhibitors of the IKK complex. It will be appreciated that compounds of this invention can exhibit IκB kinase inhibitor activities of varying degrees. Following assay procedures described herein, the IκB kinase inhibition average IC₅₀ values for the inventive compounds were generally below about 10 micromolar, preferably below about 1.0 micromolar, and more preferably below about 100 nanomolar.

Cellular Assays: A variety of cellular assays are also useful for evaluating compounds of the invention:

Multiple Myeloma (MM) Cell Lines and Patient-Derived MM Cells Isolation

RPMI 8226 and U266 human MM cells are obtained from American Type Culture Collection (Manassas, Va.). All MM cell lines are cultured in RPMI-1640 containing 10% fetal bovine serum (FBS, Sigma-Aldrich Co., St. Louis, Mo.), 2 mM L-glutamine, 100 U/mL penicillin (Pen) and 100 μg/mL streptomycin (Strep) (GIBCO brand cell culture products available from Invitrogen Life Technologies, Carlsbad, Calif.). Patient-derived MM cells are purified from patient bone marrow (BM) aspirates using ROSETTESEP (B cell enrichment kit) separation system (StemCell Technologies, Vancouver, Canada). The purity of MM cells are confirmed by flow cytometry using PE-conjugated anti-CD138 antibody (BD Biosciences, Bedford, Mass.).

Bone Marrow Stroma Cell Cultures

Bone marrow (BM) specimens are obtained from patients with MM. Mononuclear cells (MNCs) separated by Ficoll-Hipaque density sedimentation are used to establish long-term BM cultures as previously described (Uchiyama et al., Blood 1993, 82, 3712-3720). Cells are harvested in Hank's Buffered Saline Solution (HBSS) containing 0.25% trypsin and 0.02% EDTA, washed, and collected by centrifugation.

Cell Proliferation Via Measurement of DNA-Synthesis Rate

Proliferation is measured as described (Hideshima et al., Blood 2000, 96, 2943). MM cells (3×10⁴ cells/well) are incubated in 96 well culture plates (Corning Life Sciences, Corning, N.Y.) in the presence of media or an IKK inhibitor of this invention for 48 h at 37° C. DNA synthesis is measured by [³H]-thymidine ([³H]-TdR, New England Nuclear division of Perkin Elmer Life and Analytical Sciences, Boston, Mass.) incorporation into dividing cells. Cells are pulsed with [³H]TdR (0.5 μCi/well) during the last 8 h of 48 h cultures. All experiments are performed in triplicate.

MTT Cell Viability Assay

The inhibitory effect of the present compounds on MM growth is assessed by measuring the reduction of yellow tetrazolium MTT (3-(4,5-dimethylthiazolyl-2)-2,5-diphenyltetrazolium bromide) by metabolically active cells (J. Immunol. Methods 1994, 174, 311-320). Cells from 48 h cultures are pulsed with 10 μL of 5 mg/mL MTT to each well for the last 4 h of the 48 h cultures, followed by 100 μL isopropanol containing 0.04N HCl. Absorbance is measured at 570 mm using a spectrophotometer (Molecular Devices Corp., Sunnyvale Calif.).

NF-κB Activation via Electrophoretic Mobility Shift Assay

Electrophoretic mobility shift analyses (EMSA) are carried out as described (Hideshima et al., Oncogene 2001, 20, 4519). Briefly, MM cells are pre-incubated with an IKK inhibitor of this invention (10 μM for 90 min) before stimulation with TNF-α (5 ng/mL) for 10 to 20 min. Cells are then pelleted, resuspended in 400 μL of hypotonic lysis buffer (20 mM HEPES, pH 7.9, 10 mM KCl, 1 mM EDTA, 0.2% Triton X-100, 1 mM Na₃VO₄, 5 mM NaF, 1 mM PMSF, 5 μg/mL leupeptin, 5 μg/mL aprotinin), and kept on ice for 20 min. After centrifugation (14000 g for 5 min) at 4° C., the nuclear pellet is extracted with 100 μL hypertonic lysis buffer (20 mM HEPES, pH 7.9, 400 mM NaCl, 1 mM EDTA, 1 mM Na₃VO₄, 5 mM NaF, 1 mM PMSF, 5 μg/mL leupeptin, 5 μg/mL aprotinin) on ice for 20 min. After centrifugation (14000 g for 5 min) at 4° C., the supernatant is collected as nuclear extract. Double-stranded NF-κB consensus oligonucleotide probe (5′-GGGGACTTTCCC-3′, Santa Cruz Biotechnology Inc., Santa Cruz Calif.) is end-labeled with [(³²P]ATP (50 μCi at 222 TBq/mM; New England Nuclear division of Perkin Elmer Life and Analytical Sciences, Boston, Mass.). Binding reactions containing 1 ng of oligonucleotide and 5 μg of nuclear protein are conducted at room temperature for 20 min in a total volume of 10 μL of binding buffer (10 mM Tris-HCl, pH 7.5, 50 mM NaCl, 1 mM MgC₂, 0.5 mM EDTA, 0.5 mM DTT, 4% glycerol (v/v), and 0.5 μg poly (dI-dC) (Amersham Biosciences AB, Uppsala, Sweden). For supershift analysis, 1 μg of anti-p65 NF-κB Ab is added 5 min before the reaction mixtures, immediately after addition of radiolabeled probe. The samples are loaded onto a 4% polyacrylamide gel, transferred to Whatman paper (Whatman International, Maidstone, U.K.), and visualized by autoradiography.

Diffuse Large B-Cell Lymphoma (DLBCL) Cell Proliferation Assay

ABC-like (LY3 and Ly10) and GCB-like (Ly7 and Ly19) DLBCL cell lines (Alizadeh et al., Nature 2000, 403, 503-511; Davis et al., J. Exp. Med. 2001, 194, 1861-1874) are maintained in growth medium (GM, Iscove's DMEM+10% FBS) by passaging cells twice per week. Cells are starved overnight in Iscove's DMEM medium +0.5% FBS overnight before being plated in the proliferation assay. On the day of the assay, cells are counted and viability is checked using Trypan Blue staining. For the Ly3 and Ly10 cells, 5000 cells are plated in GM per well in a 96-well plate. The Ly7 and Ly19 cells are plated at 10,000 cells per well. IKK inhibitors are first dissolved in DMSO and then diluted in GM to reach the final concentrations of 80 μM-0.01 μM. Each concentration is plated in triplicate. Cell viability is determined using a standard WST-1 cell viability assay (Roche Applied Science, Indianapolis, Ind.).

Human Peripheral Blood Monocyte (PBMC) Cytokine Release Assay

Human PBMC is purified from normal donor whole blood by Ficoll gradient method. After a PBS wash, PBMC are re-suspended in AIM-V medium. Serially diluted IKK inhibitors of this invention in 100% DMSO are added at 1 μl to the bottom of a 96-well plate and mixed with 180 μl 4.5×10⁵ PBMC in AIM-V media per well. After preincubating PBMC with inhibitor at 37° C. for 40 min, cells are stimulated with 20 μl of either LPS (100 ng/ml) or anti-CD3 (0.25 μg/ml) and anti-CD28 (0.25±1 g/ml) (Pharmingen division of BD Biosciences, Bedford, Mass.) at 37° C. for 5 hours. The supernatants are collected and assessed for IL-1β or TNF-α release using standard commercially available ELISA kits.

Human Chondrocyte Matrix Metalloproteases (MMPs) Release Assay

Human chondrocyte cell line SW1353 (ATCC, Manassas, Va.) is cultured containing 10% fetal bovine serum (Hyclone, Logan, Utah), 2 mM L-glutamine (GIBCO brand cell culture products available from Invitrogen Life Technologies, Carlsbad, Calif.) and 1% Pen/Strep (GIBCO). Cells are seeded in 96-well Poly-D-Lysine plate (BD BICCOAT, Black/Clear bottom, BD Biosciences, Bedford, Mass.). Serially diluted IKK inhibitors at 1 μl are added to each well of 96-well plates and mixed with 180 μl 4.5×10⁵ chondrocytes per well. After pre-incubating cells with compounds for 1 hr at 37° C., cells are stimulated with 20 μl IL-1β (10 ng/mL, R&D Systems Inc.) at 37° C. for 24 hrs. The supernatants are then collected and assessed for production of matrix metalloproteinases (MMPs) using commercially available ELISA kits.

Human Fibroblast Like Synoviocyte (HFLS) Assay

HFLS isolated from RA synovial tissues obtained at joint replacement surgery are provided by Cell Applications Inc. (San Diego, Calif.). IKK inhibitors of the invention are tested for their ability to block the TNF- or IL-1β-induced release of IL-6 or IL-8 from these cells using commercially available ELISA kits. Cell culture conditions and assay methods are described in Aupperle et al., Journal of Immunol. 1999, 163, 427-433.

Human Cord Blood Derived Mast Cell Assay

Human cord blood is obtained from Cambrex (Walkersville, Md.). Mast cells are differentiated and are cultured in a manner similar to that described by Hsieh et al., 1. Exp. Med. 2001, 193, 123-133. IKK inhibitors of the invention are tested for their ability to block the IgE- or LPS-induced TNFα release using commercially available ELISA kits.

Osteoclast Differentiation and Functional Assays

Human osteoclast precursors are obtained as cryopreserved form from Cambrex (Walkersville, Md.). The cells are differentiated in culture based on instructions from the manufacturer. IKK inhibitors of the invention are tested for their ability to block the differentiation, bone resorption and collagen degradation as described previously (see Khapli et al., Journal of Immunol. 2003, 171, 142-151; Karsdal et al., J Biol. Chem. 2003, 278, 44975-44987; Takami et al., Journal of Immunol. 2002, 169, 1516-1523).

Rat Models for Rheumatoid Arthritis

Such testing is known in the literature and include a standard rat LPS model as described in Conway et al., “Inhibition of Tumor Necrosis Factor-α (TNF-αProduction and Arthritis in the Rat by GW3333, a Dual Inhibitor of TNF-Converting Enzyme and Matrix Metalloproteinases”, J. Pharmacol. Exp. Ther. 2001, 298(3), 900-908; a rat adjuvant induced arthritis model as described in Pharmacological Methods in the Control of Inflammation (1989) p 363-380 “Rat Adjuvant Arthritis: A Model of Chronic Inflammation” Barry M. Weichman {author of book chapter; Alan R. Liss Inc Publisher}; and a rat collagen induced arthritis model as described in Pharmacological Methods in the Control of Inflammation (1989) p 395413 “Type II Collagen Induced Arthritis in the Rat” D E Trentham and R A Dynesuis-Trentham {authors of book chapter; Alan R. Liss Inc Publisher}. See also, “Animal Models of Arthritis: Relevance to Human Disease” by Bendele et al., Toxicologic Pathology 1999, 27(1), 134-142.

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example.

1. A compound of formula (II): 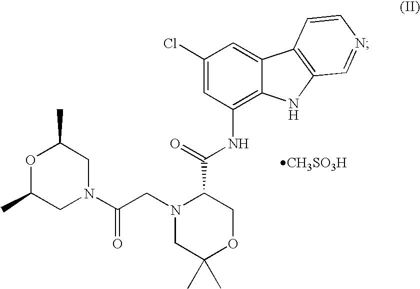
or crystalline forms thereof.

2. The compound of formula (II) according to claim 1, wherein the crystalline form is substantially crystalline.

3. The compound of formula (II) according to claim 1, wherein the crystalline form is Form 1, characterized by at least one of the X-ray powder diffraction peaks at 20 angles of 4.619°, 13.652°, 17.834°, 19.225°, 20.728°, 23.711°, and 25.212°.

4. The compound of formula (II) according to claim 1, wherein the crystalline form is Form 1, characterized by at least one of the X-ray powder diffraction peaks shown in Table 1.

5. The compound of formula (II) according to claim 1, wherein the crystalline form is Form 1, characterized by an X-ray powder diffraction pattern substantially similar to FIG. 1.

6. The compound of formula (II) according to claim 1, wherein the crystalline form is Form 1, characterized by at least one of the following features:
(I-i) at least one of the X-ray powder diffraction peaks shown in Table 1.
(I-ii) an X-ray powder diffraction pattern substantially similar to FIG. 1.
(I-iii) a differential scanning calorimetry (DSC) profile having an endotherm range of about 215° C. to about 250° C.

7. The compound of formula (II) according to claim 1, wherein the crystalline form is Form 2, characterized by at least one of the X-ray powder diffraction peaks at 20 angles of 3.694°, 11.163°, 15.551°, 18.737°, 20.183°, 23.001°, and 23.776°.

8. The compound of formula (II) according to claim 1, wherein the crystalline form is Form 2, characterized by at least one of the X-ray powder diffraction peaks shown in Table 2.

9. The compound of formula (II) according to claim 1, wherein the crystalline form is Form 2, characterized by an X-ray powder diffraction pattern substantially similar to FIG. 5.

10. The compound of formula (II) according to claim 1, wherein the crystalline form is Form 2, characterized by at least one of the following features:
(II-i) at least one of the X-ray powder diffraction peaks shown in Table 2.
(II-ii) an X-ray powder diffraction pattern substantially similar to FIG. 5.
(II-iii) a differential scanning calorimetry (DSC) profile showing a endotherm range of about 120° C. to about 170° C.

11. A pharmaceutical composition comprising a pharmaceutically effective amount of a compound according to claim 1, and a pharmaceutically acceptable carrier.

12. A method for treating a patient suffering from, or subject to, a pathological condition capable of being ameliorated by inhibiting IKK-2 comprising administering to said patient a pharmaceutically effective amount of the compound according to claim 1.

13. A method for treating a patient suffering from, or subject to, an inflammatory disease or immune-related disease comprising administering to said patient a pharmaceutically effective amount of the compound according to claim 1.

14. The method of claim 13, wherein the disease is rheumatoid arthritis, psoriasis, inflammatory bowel disease, chronic obstructive pulmonary disease (COPD) or COPD exacerbations.

15. A method for treating a patient suffering from, or subject to, cancer comprising administering to said patient a pharmaceutically effective amount of the compound according to claim 1.

16. A method for treating a patient suffering from rheumatoid arthritis, comprising administering to the patient a pharmaceutically effective amount of the compound according to claim 1.
